(19) United States
(12) Patent Application Publication
(10) Pub. No.: US 2023/0233748 A1
(43) Pub. Date: Jul. 27, 2023

(54) METHODS AND DEVICES FOR PLACEMENT OF SUBDURAL DRAINS AND OTHER MEDICAL DEVICES WITHIN A CRANIUM

(71) Applicant: KENNETH R. STUEBE, ALACHUA, FL (US)

(72) Inventor: KENNETH R. STUEBE, ALACHUA, FL (US)

(21) Appl. No.: 17/585,691

(22) Filed: Jan. 27, 2022

(51) Int. Cl. A61M 1/00

(52) U.S. Cl. CPC A61M 1/84; A61M 2210/0693; A61M 2205/0272

(57) ABSTRACT

Methods and devices for positioning of subdural drain within an evacuated hematoma cavity. Embodiments include a tethered subdural drain with a puck attached to a leading end of a tether and a subdural drain attached to the terminal end of the tether. The puck is placed in vivo through a first hole in a cranium. A ex vivo guide manipulates the puck and tether through an evacuated hematoma cavity to be retrieved through a second hole in the cranium. Pulling the tether through the second hole advances the subdural drain attached thereto through the first hole and into the hematoma cavity.

### BACKGROUND OF INVENTION

Cranial hematomas are an accumulation of blood within the cranium, usually as a result of head trauma or injury. Arteries and veins between the brain and the cranium are broken or torn causing blood to accumulate inside the cranium, between the dura matter and the arachnoid matter of the brain (subdural hematoma) or between the dura matter and the cranium (epidural hematoma). The accumulated blood forms the cranial hematoma which exerts pressure on the brain tissue and can lead to further tearing of neighboring arteries and veins, causing additional bleeding and expansion of the hematoma. This pressure can inhibit perfusion to the brain and can result in permanent injury to the brain tissue. Effective treatment of a cranial hematoma is necessary to minimize expansion of the hematoma and the resulting brain injury.

One option for treating a cranial hematoma is evacuation of the hematoma by creating several holes in the cranium and membranes to reduce pressure on the brain. The cranial hematoma can be drained and evacuated through the holes. Typically, a sterile fluid, such as normal saline, is introduced into one hole while suction is simultaneously applied to the other hole. With this technique, the fluid flushes out the hematoma material and relieves the pressure on the brain. Once the hematoma is evacuated, a surgical drain, such as a subdural drain, is often inserted into the subdural space to maintain drainage in the space and prevent further build-up of pressure.

A subdural drain comprises a tube with multiple ports to drain fluid from within the evacuated subdural space. Placement of the subdural drain must be done quickly, as the subdural space created by the hematoma will be filled by the brain as perfusion to the brain resumes. The current process of inserting a subdural drain requires blind insertion of the distal end of the subdural drain through one of the holes and manually pushing the drain into the cranium. Ideally the subdural drain can be positioned in the area where the hematoma formed. It is difficult, however, to position a subdural drain accurately during blind insertion because of the curvature of the cranium, the narrowness of the subdural space and the presence of bridging arteries and veins within the subdural space. Thus, the outcome of placement of a subdural drain can be unpredictable and often placing the subdural drain even near the hematoma area is the best achievable outcome. Injury to the delicate brain tissue during a blind insertion of a subdural drain is a constant concern for the surgeons.

There is a need in the medical arts for methods and devices that allow for safe placement of subdural drains, which are minimally invasive and more accurate than the current blind insertion techniques.

### BRIEF SUMMARY

The subject invention provides medical devices and methods by which a surgical drain can be safely and accurately positioned using two prepared holes within a cranium. More specifically, the subject invention provides medical devices and methods for safe and accurate in vivo positioning of a subdural drain. Embodiments of the medical devices of the subject invention utilize an elongated flexible tether that can be attached at one end to a subdural drain, and at another end to a magnetic “puck.” The puck can be inserted into a first hole in the cranium. A magnetic guide can be placed ex vivo the cranium, and in proximity to the first hole and near or against the cranium, to allow the magnetic forces to attract the inserted puck through the tissue and cranium. Starting next to the first hole, the magnetic guide, with the now in vivo puck attracted thereto through the cranium, is moved or directed in a controlled manner over the cranium and towards the second hole in the cranium. As the magnetic guide is moved or directed over the cranium, the puck, now located in the subdural space, follows and drags or pulls the tether along with it towards the second hole. When the magnetic guide reaches the second hole, the puck attracted thereto, is pulled out of the second hole by the magnetic guide along with the end of the tether.

The subdural drain attached to the other end of the tether, can then be inserted into the first hole and, by pulling the tether now extending through the second hole, the subdural drain, can be pulled through the first hole, into the subdural space, and towards the second hole while being guided or directed by the tether. A tether can also be a separate component from the subdural drain. After the tether is guided between the two holes, the subdural drain can be operably attached to the end of the tether extending from the first hole. Alternatively, the magnetic puck can be removed from the positioned tether and the subdural drain attached to that end of the tether. Now with the subdural drain attached, the subdural drain can then be pulled back into the second hole, into the subdural space, and towards the first hole by pulling the tether end extending through the first hole. Ideally, the subdural drain will follow substantially the same path as the tether that was pulled by the puck. Where a subdural drain is utilized with embodiments of the subject invention, the subdural drain can have a tapered distal end that can flex to facilitate entry through the first hole and/or in vivo movement.

Embodiments of the subject invention can also be beneficial in inserting and placing other medical devices, such as medicament delivery devices, stimulators, optical devices, and other apparatuses, which may be utilized to treat or examine a subdural hematoma or the space created thereby. With the devices and techniques of the subject invention, a tether can be extended through two holes in a cranium that were utilized to drain a subdural hematoma. The magnetic guide can pull the puck, inserted into the first hole, through the evacuated subdural space, creating a straight path between the two holes that can be subsequently followed by the medical device attached to the terminal end of the tether. Utilizing the tether extending between the two holes, the medical device can be pulled under the cranium and directly into the subdural space left by the evacuated cranial hematoma. The subject invention allows a subdural drain, or other medical device, to be quickly and optimally inserted while inhibiting additional, unnecessary tissue injury.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variation of these dimensions that will allow the subject invention to function for its intended purpose are considered to be within the scope of the subject invention.

FIG. 1 is an illustration of an embodiment of a subdural drain, according to the subject invention, being positioned between two holes within a cranium.

FIG. 2A is an illustration of an embodiment of the subdural drain shown in FIG. 1, optimally positioned between the two holes and within the subdural space.

FIG. 2B is an illustration of an embodiment of the subdural drain shown in FIG. 1, prior to positioning of the subdural drain, with the tether traversing between the first hole and the second hole in a cranium.

FIGS. 3A and 3B illustrate an embodiment of a tethered subdural drain with a tapered tip (FIG. 3A) attached to a tether, according to the subject invention. The tapered tip can facilitate insertion of the subdural drain into a first hole (FIG. 3B) and, in a similar manner, the subsequent exit from a second hole. The tether can be removed, after placement of the subdural drain attached thereto, by cutting the distal end from the subdural drain.

FIG. 4 illustrates an alternative embodiment of a tethered subdural drain with a rounded tip attached to a tether, according to the subject invention. The tether can be removed, after placement of the subdural drain attached thereto, by cutting the distal end from the subdural drain.

FIG. 5 illustrates another alternative embodiment of a tethered subdural drain with a rounded plug tip attached to a tether, according to the subject invention. The tether can be removed, after placement of the subdural drain attached thereto, by removing the plug from the subdural drain or cutting the distal end of subdural drain to remove the plug end.

FIG. 6 illustrates the position of the tether relative to a subdural drain, wherein the terminal end of the tether is operably attachable or fixedly attached to the distal end of the subdural drain.

FIGS. 7A, 7B, and 7C illustrate embodiments of various diameters of a tether, according to the subject invention.

FIG. 8 illustrates embodiments of the attractive surface area on three different configurations of a magnetic puck, according to the subject invention.

FIG. 9 illustrates an embodiment of an introducer, according to the subject invention.

FIG. 10 is a flowchart showing an embodiment of a method for inserting a tethered subdural drain, according to the subject invention.

FIG. 11 is a flowchart showing an alternative method of operably attaching the tether to a surgical drain.

FIG. 12 is a flowchart showing an alternative method of removing the puck from the tether and operably attaching the tether to a surgical drain.

### DETAILED DISCLOSURE

The subject invention pertains, in general, to a medical device and methods for insertion and in vivo placement of a surgical drain within a cranium by utilizing a tether. More specifically, the subject invention provides one or more embodiments of a tethered subdural drain, that can be placed by utilizing one or more magnetic components to manipulate the tether through a subdural space in a cranium and utilizing the tether to pull and guide a subdural drain into or through the subdural space utilizing two openings within the cranium.

The subject invention is particularly useful when subdural drains are utilized to treat and/or drain a cranial hematoma. While the subject application describes, and many of the terms herein relate to, a use for placement of a subdural drain in a subdural space, a person of skill in the art will recognize that the subject invention is not limited to tethered subdural drains or limited to methods of placing a tethered subdural drain within a cranium.

In the description that follows, a number of terms are utilized. In order to provide a clear and consistent understanding of the specification and claims, the following definitions are provided.

The term “patient” as used herein, describes an animal, including mammals to which the systems and methods of the present invention are applied. Mammalian species that can benefit from the disclosed systems and methods include, but are not limited to, apes, chimpanzees, orangutans, humans, and monkeys; domesticated animals (e.g., pets) such as dogs, cats; veterinary uses for large animals such as cattle, horses, goats, and sheep; and any wild or non-domesticated animal for veterinary or tracking purposes.

The term “surgeon” as used in the subject invention is merely for literary convenience. The term should not be construed as limiting in any way. The devices, apparatuses, methods, techniques and/or procedures of the subject invention could be utilized by any person desiring or needing to do so and having the necessary skill and understanding of the invention.

The term “about” is used herein to describe some quantitative aspects of the invention, for example, the length of a tether or the diameter of a puck. It should be understood that absolute accuracy is not required with respect to those aspects for the invention to operate. When the term “about” is used to describe a quantitative aspect of the invention, the relevant aspect may be varied by up to ±10% (e.g., ±1%, ±2%, ±3%, ±4%, ±5%, ±6%, ±7%, ±8%, ±9%, and/or ±10% or by ±a percentage between any two of the listed values.

Also, as used herein, and unless otherwise specifically stated, the terms “operable communication,” “operable connection,” “operably connected,” “cooperatively engaged” and grammatical variations thereof mean that the particular elements are connected in such a way that they cooperate to achieve their intended function or functions. The “connection” or “engagement” may be direct, or indirect, physical or remote.

It is to be understood that the figures and descriptions of embodiments of the present invention have been simplified to illustrate elements that are relevant for a clear understanding of the invention, while eliminating, for purposes of clarity, other elements that may be well known. Those of ordinary skill in the art will recognize that other elements may be desirable and/or required in order to implement the present invention.

Throughout the application, reference is made to the leading end of a tether and the terminal end of a tether. The leading end is the end to which a puck is attached and is inserted or placed within the first hole. The terminal end is the end that attaches to a subdural drain.

Reference is also made throughout the application to the “distal end” and “proximal end” of a subdural drain. As used herein, the distal end is that end that is attached to the terminal end of the tether. For example, the distal end is that end that is inserted into a first hole and that extends from a second hole. Conversely, the proximal end is that end of the subdural drain that is furthest from the tether. For example, the proximal end of a subdural drain can extend from the first hole in the cranium.

The present invention is more particularly described in the following examples that are intended to be illustrative only because numerous modifications and variations therein will be apparent to those skilled in the art. As used in the specification and in the claims, the singular for “a,” “an” and “the” include plural referents unless the context clearly dictates otherwise.

The transitional term “comprising,” which is synonymous with “including” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

Reference will be made to the attached Figures on which the same reference numerals are used throughout to indicate the same or similar components. With reference to the attached Figures, it can be seen that embodiments of a medical device of the subject invention pertain to a tethered surgical drain 80 that comprises an elongated tether 100 with a terminal end 15 and a leading end 20. At the leading end of the tether there can be fixedly attached a puck 150 that can attract or can be attracted to a guide 200. At the terminal end of the tether there can be fixedly attached or operably attachable a surgical drain or other type of cranial drain or intracranially-placed medical device. In a specific embodiment, a subdural drain 300 is fixedly attached to the terminal end of the tether, such that the tether is utilized with the subdural drain attached. Each of these general components can have one or more sub-components, which will be discussed in detail below.

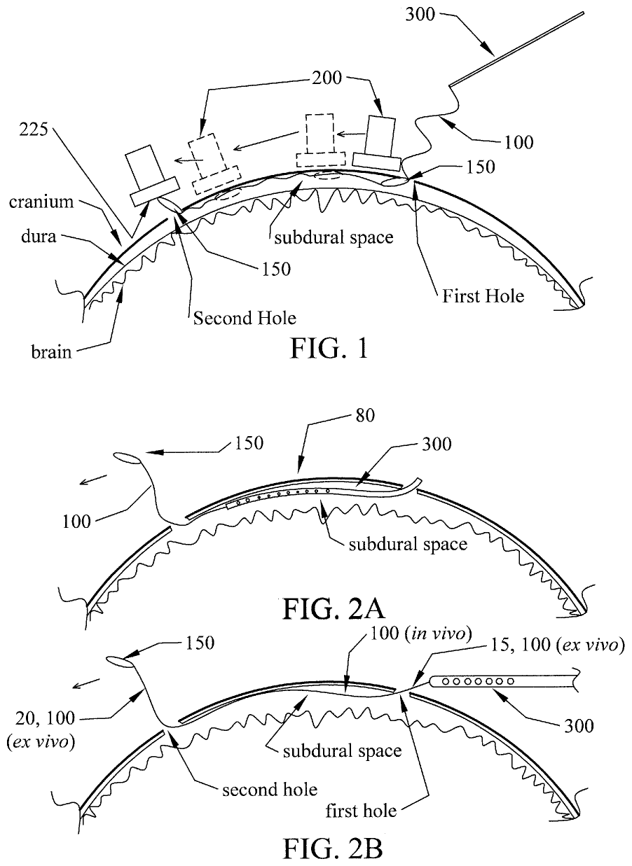

The embodiments of the subject invention are most advantageously utilized between at least two holes, a first hole and a second hole, prepared within a cranium, as shown for example in FIGS. 1 and 2A. Additional holes can be utilized to assist positioning, as alternatives if one of the first or second holes is not usable, or for later adjustment of the position of a subdural drain. The holes can be located anywhere on the cranium, but are, preferably, of a size that permits passage of a tethered subdural drain 80 therethrough and are distanced as necessary or feasible to allow placement of the subdural drain 300 therebetween, as described herein. By way of example, a first hole and a second hole can refer to burr holes, or any other type of hole, prepared in the cranium of a patient.

A tether 100 utilized with embodiments of the subject invention can have a length that is limp, bendable, pliable, flexible, or otherwise capable of conforming, thus capable of being inserted into a hole and being directed around objects, material, and obstructions within a cranium. The circumferential shape of a tether can be flat or tape-like, square, rectangular, circular, cylindrical, oval, or some variation thereof. A tether can also have one or more sufficiently low friction surfaces to facilitate sliding across in vivo structures and objects, but that does not inhibit manual manipulation. As will be discussed in further detail below, a tether can be used to manually pull a subdural drain through a subdural space between two holes in a cranium. It can be important to control movement and direction of the subdural drain as it moves. As such, a tether comprising a non-elastic material or one with minimal elasticity can be beneficial to inhibit uncontrolled or sudden movements, which can translate to the movement of the subdural drain. A tether can also transmit tactile information or feedback to a surgeon regarding the position of the subdural drain and/or obstructions encountered by the subdural drain that may not have been encountered by the puck. In summary, it can be advantageous for a tether to have (a) sufficient conformability or flexibility, (b) a low-friction surface, and (c) and be manually manipulable. It can be further advantageous for the tether to comprise one or more materials that provide adequate tensile strength to (a) allow pulling of the subdural drain through the subdural space in a cranium, (b) transmit tactile information to the surgeon, and (c) minimize elasticity. Sutures are known in the art and can comprise any one or more natural or man-made sterilizable materials suitable for a tether of the subject invention. FIGS. 1, 3A, 4, and 5 illustrate non-limiting examples of a tether utilized with a subdural drain, according to the subject invention.

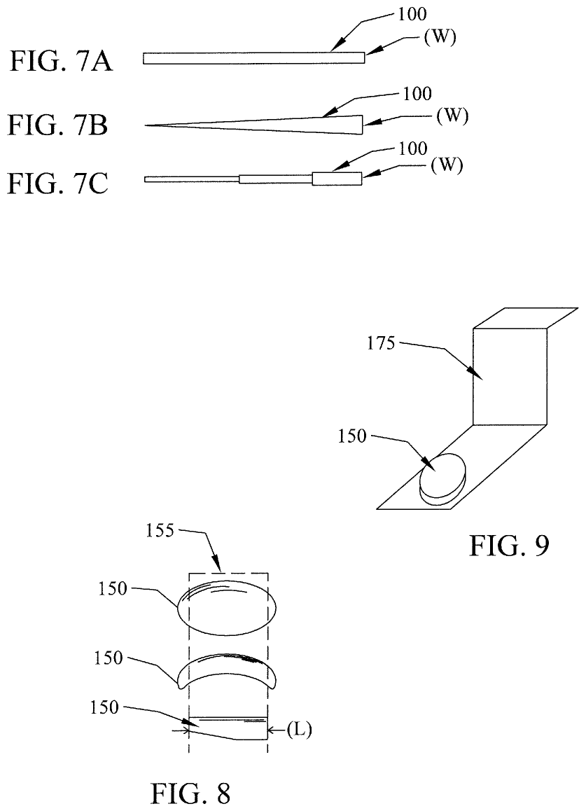

The width of the material utilized for a tether 100 can depend upon a variety of factors, including, but not limited to, the material utilized, the size of the subdural drain 300 and/or the puck to which the tether is attached, whether the tether will be removed from the subdural drain and/or the puck after placement, and other factors that would be understood by those with skill in the art. In one embodiment, a tether has a substantially consistent width between where it attaches to a subdural drain and where it attaches to a puck. FIG. 7A illustrates an example of this embodiment. In more particular embodiment, a tether has a width (W) of at least about 0.01 mm, 0.02 mm, 0.03 mm, 0.04 mm, 0.05 mm, 0.06 mm, 0.07 mm, 0.08 mm, 0.09 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1.0 mm, 1.5 mm, 2.0 mm, 2.5 mm and/or a width between any two of the listed values.

Suture thread can be a suitable material for a tether. Suture thread can comprise different natural and man-made materials that provide a consistent width along the length and impart strength and minimal elasticity. Suture thread is also manipulable and sterilizable and is commonly utilized in association with other medical devices, including subdural drains. In one embodiment, a tether comprises a length of suture thread.

Alternatively, the width (W) of a tether 100 can be inconsistent between where a tether 100 attaches to a subdural drain 300 and to a puck 150. In one embodiment, the width of a tether decreases from the terminal end 15 to the leading end 20. In a further embodiment, the width decreases incrementally towards the leading end 20, such that the tether narrows along the length between where the tether attaches to the subdural drain and to the puck, as shown, for example, in FIG. 7B. In an alternative embodiment, the width decreases stepwise towards the leading end, between where the tether attaches to the subdural drain and to the puck, as shown, for example, in FIG. 7C. In a specific embodiment, a tether has a length that varies in width between at least two of the above-listed values.

At the leading end 20 of a tether 100 there can be a fixedly attached puck 150, as shown, for example, in FIG. 6. A puck is essentially a transport mechanism that carries a tether from a first hole to a second hole in a cranium. The shape and dimensions of a puck can vary depending on a variety of factors including, but not limited to, the tether material, the in vivo distance to be traveled, the location of use, possible in vivo obstructions, and other factors understood by those with skill in the art. For example, a puck can carry a tether from a first burr hole in a cranium to a second burr hole in a cranium through a subdural space created by the evacuation of a cranial hematoma. By way of a more specific example, a puck can carry a tether through a first burr hole in a cranium through a subdural space created by the evacuation of a subdural hematoma and out of a second hole in the cranium, as shown, in the example in FIG. 2B. There are typically few obstructions in this type of space and any obstructions can often be bypassed by the puck. A puck can have any of a variety of shapes from disk-like to spherical, cylindrical to frustoconical, curved or flat, as well other shapes and variations therebetween. In one embodiment, the puck has a disk-shaped circumference, such as shown, for example, in FIG. 9, having a diameter of between about 1 mm and about 40 mm, between about 10 mm and about 30 mm, or between about 15 mm and about 25 mm. In an alternative embodiment, the puck a circumferential shape that varies in diameter, but the largest measurable length (L) is between about 1 mm and about 40 mm, between about 10 mm and about 30 mm, or between about 15 mm and about 25 mm. For example, a puck can have a wedge shape, such as shown in FIG. 8 (bottom), such that the largest measurable length would be from the end of the wedge to the opposite flat end.

A puck 150 can be manipulated, directed, or moved in vivo by using an ex vivo guide 200 placed on or near the cranium. One example of this manipulation is shown in FIG. 1. In one embodiment, a guide is a hand-held device with a contact surface 225 that is placed against or in proximity to a cranium. In one embodiment, the material of an in vivo placed puck and that of the contact surface of an ex vivo positioned guide have a magnetic attraction therebetween. The force of the magnetic attraction allows the guide to precisely manipulate the in vivo puck.

An important factor in designing a puck 150 is the ability to move and/or direct the puck in vivo utilizing an ex vivo guide 200. Preferably, the shape of the puck facilitates the magnetic attraction and allows the guide to move or direct the in vivo puck. The thickness of a cranium and tissue can vary the distance between an in vivo puck and an ex vivo guide as the puck moves. It can be further preferable for the magnetic attraction between the puck and the guide to be sufficient that the magnetic attraction therebetween is not lost due to changes in the distance therebetween.

In one embodiment, the puck comprises a ferromagnetic material and the guide comprises a magnet configured to attract the ferromagnetic material of the puck. The magnet utilized for a guide can be at least one of a permanent magnet, rare earth magnet, or an electromagnet.

In an alternative embodiment, the guide comprises a ferromagnetic material and the puck comprises a magnet configured to attract the ferromagnetic material of the guide. The magnet utilized for puck can be a permanent magnet or a rare earth magnet.

In a still further embodiment, both the puck 100 and the guide 200 are magnets arranged with opposing poles to achieve a magnetic attraction therebetween. In yet a further embodiment, the puck and/or the guide comprise both a magnet and a ferromagnetic material. In a specific embodiment, the materials of a puck and guide are configured so that the puck and the guide align in a specific orientation that, for example, allows the puck to move or be directed while trailing a tether. Thus, the puck can have north-south poles that form a magnetic attraction to the north-south poles of a guide causing the puck to align with the guide.

The ability to select one or more magnets and ferromagnetic materials suitable for a puck and a guide is within the skill of a person trained in the art. Because the puck can move in vivo, it can be preferable for the puck to be as small as feasible so as not to interfere with in vivo movement and to inhibit tissue damage. Thus, it can be preferable to adjust the size or the magnetic force of a guide to achieve continuous magnetic attraction to a puck. It can be equally important for the puck to have an attractive surface area 155, as illustrated, for example, in FIG. 8, of sufficient size to be sufficiently magnetically attracted to the guide. As mentioned above, a puck can have any of a variety of outward shapes or configurations. In a specific embodiment, the puck has an attractive surface area 155 sufficient to create and maintain a magnetic attraction to the guide, regardless of the outward shape or configuration of the puck. In one embodiment, the attractive surface area 155 of a puck has a minimum diameter that is at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 30 mm, and/or a diameter between any two of the listed values.

An important first step in utilizing embodiments of a tethered subdural drain can be the initial insertion of the puck 150 into a first hole. While the magnetic attraction between the puck and the guide 200 can ensure that the puck is properly aligned, it can be beneficial if the puck is inserted into the first hole in a manner that inhibits turning, flipping, or rotation of the puck when in vivo. In one embodiment, the puck can be dropped or delivered into and/or through a first hole with a introducer 175, an example of which is shown in FIG. 9. In one embodiment, the introducer comprises a flat surface that supports the puck in the correct alignment for insertion into a first hole. In a further embodiment, the puck is temporarily secured to the introducer, such that when deposited into and/or through the first hole, the magnetic attraction of the guide can remove the puck from the introducer and into a fully in vivo position. By way of a non-limiting example, the introducer can have a magnetic attraction to the puck, wherein the magnetic attraction is less than the magnetic attraction of the guide. In another non-limiting example, the drop introducer can have a shape that is complimentary to the shape of a puck, such that a puck placed on the introducer is inhibited from sliding off the end while being deposited into and/or through the first hole. As the ordinary artisan can readily envision, an introducer can have other features that can temporarily support a puck thereon.

The embodiments of the subject invention are advantageous for the placement of a surgical drain in a cranial space. More particularly, embodiments of the subject invention are advantageous for placement of a subdural drain within a space created in a cranium by an evacuated subdural hematoma. Subdural drains are frequently used for a variety of medical reasons to drain blood, occlusions, and other material from the cranium. Typically, a subdural drain is a tube with multiple perforations that is placed within the cranium where fluids need to be removed as they accumulate. Embodiments of the subject invention can be advantageously incorporated with existing subdural drains.

Embodiments of the subject invention incorporate a tether 100 and puck 150 with a subdural drain. The terminal end 15 of a tether can be fixedly attached or can be operably attachable to a distal end 10 of a conventional subdural drain, an example of which is illustrated in FIG. 1. As the tether is pulled through the second hole, the distal end 10 of the subdural drain can be drawn towards and inserted into the first hole. Continued pulling of the tether through the second hole can advance the subdural drain while simultaneously guiding it into the evacuated hematoma space or cavity to be drained. In some instances, the movement of the subdural drain can push or force tissue aside while being advanced into the in vivo location or hematoma space.

The combined tether 100 and puck 150 can be fixedly attached to a subdural drain 300 to provide a single, unitary device, such as shown, for example, in FIG. 6. The tether can be utilized with the fixedly attached subdural drain. Alternatively, the combined tether and puck can be separate from the subdural drain, such that the tether can be utilized and the subdural drain later operably attached to the terminal end of the tether. In a further alternative, the puck can be removed from the leading end 20 after the tether is positioned between the first hole and the second hole. A subdural drain can then be attached to the leading end of the tether where the puck was attached and the tether pulled from the first hole back through the second hole pulling the subdural drain through the second hole and into the subdural space. The tether can be attached to a subdural drain by any of a variety of techniques and devices known in the art. For example, the tether can be attached to the end of a subdural drain with one or more sutures. By way of another example, a subdural drain can have one or more pre-made openings or ports in the distal end through which an end of the tether can be tied.

Often the distal end 10 of a typical subdural drain is a flat surface that opens to the lumen of the subdural drain. In an embodiment of the subject invention, the distal end of a subdural drain can be rounded to facilitate insertion into the first hole and advancement through the subdural space. With this embodiment, a tether can be fixedly attached to the rounded end 310. In one embodiment, the material of a subdural drain can be molded, formed, or otherwise modified to form a rounded distal end, such as shown, for example, in FIG. 4. In an alternative embodiment, the terminal end 15 of a tether 100 can be fixedly attached to a cap with a rounded exterior. The rounded 340 cap can be inserted into and/or fixedly attached to the distal end of a subdural drain. An example of this embodiment can be seen in FIG. 5.

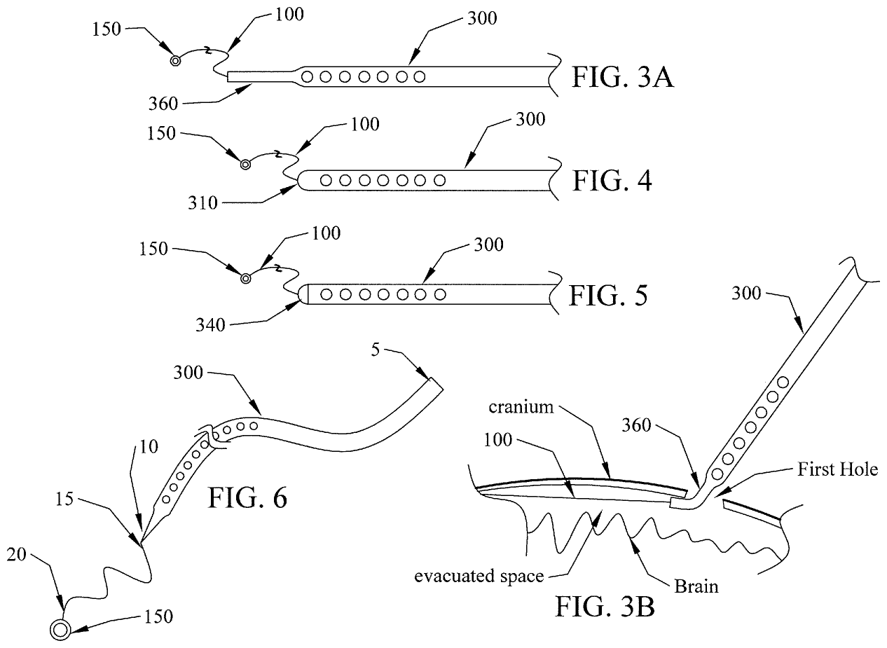

In an alternative embodiment, the distal end 15 of a subdural drain 300 can be tapered, such that the diameter of the subdural drain is smaller at the tapered end, and the tether can be fixedly attached to the tapered end 360. A non-limiting example of this embodiment is shown in FIG. 3A. A tapered tip configuration can be advantageous for insertion of the subdural drain into tight subdural spaces. For example, inserting a subdural drain through a burr hole and into a subdural space created by an evacuated hematoma can be more easily accomplished if the distal end can be turned or bent to a smaller angle than can usually be accomplished with a non-tapered subdural drain. FIG. 3B illustrates an example of a subdural drain with a tapered end and the ease with which it can be bent and inserted through a first burr hole and into the evacuated subdural space to guide the remainder of the subdural drain. In one embodiment, the tapered tip has a length of at least about 0.55 cm, 0.75 cm, 0.85 cm, 0.95 cm, 1.0 cm, 1.1 cm, 1.2 cm, 1.3 cm, 1.4 cm, 1.5 cm, 1.6 cm, 1.8 cm, 1.9 cm, 2.0 cm and/or a length between any two of the listed values. The embodiments of the subject invention are particularly advantageous for inserting an subdural drain in an evacuated hematoma cavity or space. Thus, a method for utilizing a tethered subdural drain will be described with regard to placement of a subdural drain.

Severe cranial hematomas can be treated by creating at least one, sometimes several holes around the area of the hematoma. The holes allow the hematoma to drain and can be used to inject saline or other fluids to flush the hematoma and other material out of the subdural space formed by the hematoma. Embodiments of the subject invention can be utilized when there are at least two holes.

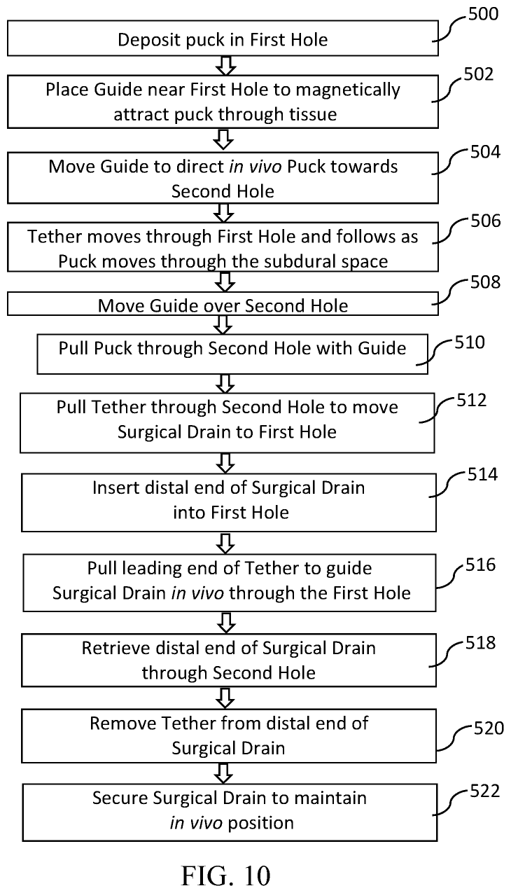

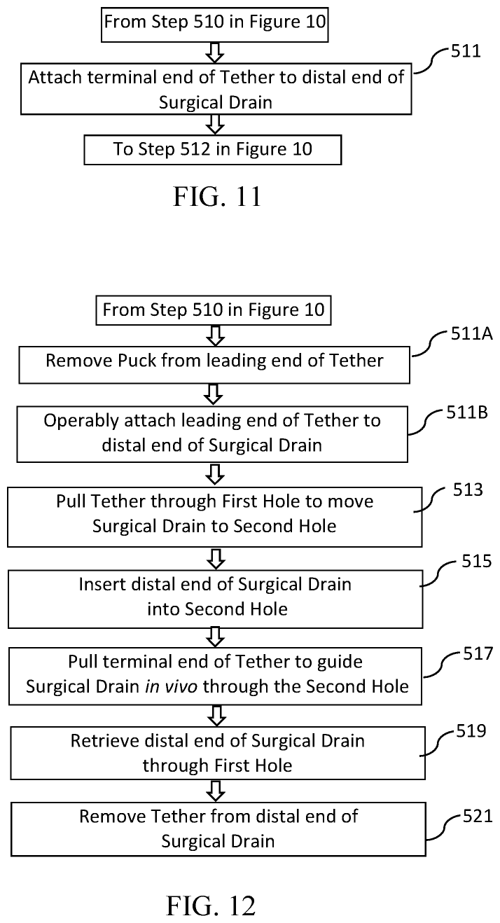

FIGS. 1, 2A, 2B, 10, 11, and 12 illustrate example methods for placement of a subdural drain through holes within a cranium. With reference to the flowchart in FIG. 10, the puck 150 attached to a tether 100 can be placed into a burr hole or first hole by hand or utilizing an introducer 175 (step 500). The magnetic guide 200 can be positioned next to or beside the first hole to initiate the magnetic attraction between the puck and the guide (step 502). Moving the magnetic guide in a controlled manner (step 504) over or near the cranium of the patient towards a second hole causes the tether to move into the first hole (step 506). Continued moving of the guide over the cranium and towards another burr hole or second hole can continue to pull or drag the puck, in a guided or directed manner, through the evacuated subdural space with the tether following through the subdural space. Upon reaching the second hole, the guide can be moved over the second hole (step 508), which causes the puck to be pulled through the second hole. At this point the puck can be retrieved along with the leading end 20 of the tether (step 510).

Next, the leading end of the tether, which now spans the subdural space between the first and second holes, can be pulled through the second hole until the distal end 10 of the subdural drain 300 is near the first hole (step 512). Once pulled into proximity to the first hole, the distal end of the subdural drain can be inserted into the first hole (step 514). The leading end of tether, still extending from the second hole, can be pulled simultaneously with the insertion of the subdural drain 516 at the first hole. The distal end of the subdural drain will bend and enter the evacuated subdural space. It will be understood at this point how a tapered or rounded distal end on the subdural drain can be beneficial in directing the drain away from the brain and into the relatively narrow subdural space. The continued and simultaneous “pulling while inserting” technique, guides the subdural drain through the subdural space in a controlled and directed manner until the distal end of the subdural drain reaches, and is retrieved, through the second hole (step 518). The tether, and any excess drain material, extending from the second hole can be removed from the distal end of the subdural drain, typically by cutting away the excess portion of the distal end of the subdural drain to which the tether is fixedly attached 520. Optionally, the surgical drain can also be secured, such as, for example, with a suture or adhesive tape, to maintain the in vivo position (step 522).

In an embodiment where the tether and puck are separate from the surgical drain, the tether and puck can be pulled through the first hole, through the subdural space, and then through the second hole, as shown, for example, in FIG. 2B. The surgical drain can be attached to the terminal end 15 of the tether, which is extending from the first hole after the leading end is pulled from the second hole (steps 500-512). Thus, as shown in the flowchart in FIG. 11, the method can include attaching the terminal end 15 of the tether to the distal end 10 of a subdural drain (step 511) and the method can continue as described above (steps 512-522). In an alternative embodiment of the method, as shown in the flowchart in FIG. 12, the puck can be removed from the leading end of the tether (step 511A) and the distal end of the surgical drain can be operably attached to the leading end to replace the puck (step 511B). With the subdural drain operably attached to the leading end of the tether, the method can proceed in substantially the same manner as shown in FIG. 2B, except that the surgical drain 300 will be pulled in the opposite direction, i.e., from the second hole to the first hole (steps 513-521). Optionally, once the surgical drain is positioned and the tether is removed from the distal end, the surgical drain can be secured to maintain the in vivo position.

Surgical drains can be effective in medical treatments. Subdural drains are a specific type of surgical drain utilized to drain a space that remains after a subdural hematoma is evacuated from the cranium. Proper placement of a subdural drain can be essential to successful recovery and healing. Conventional subdural drains are often inserted blindly through holes in the cranium, which can result in minimal control of placement and possibly brain injury. Subdural drains can be particularly problematic because of the proximity to brain. The embodiments of the subject invention provide improvements to surgical drains utilized within the cranium and specifically to subdural drains and their methods of use, which can inhibit misplacement and brain injury.

I claim:

1. A medical device, configured to be positioned in vivo, comprising components having a magnetic attraction therebetween that are utilized to direct a tether in vivo to a position between a first hole and a second hole in a cranium and utilizing the tether to guide the medical device attached thereto to the in vivo position between the first hole and the second hole.

2. A medical device, configured to be positioned in vivo, comprising:
a tether;
a puck fixedly attached to a leading end of the tether; and
a surgical drain having a distal end operably attached to a terminal end of the tether.

3. The medical device according to claim 2, wherein the surgical drain has at least one of a rounded and a tapered distal end.

4. The medical device according to claim 3, wherein the tapered distal end of the surgical drain has a length that is at least about 0.55 cm, 0.75 cm, 0.85 cm, 0.95 cm, 1.0 cm, 1.1 cm, 1.2 cm, 1.3 cm, 1.4 cm, 1.5 cm, 1.6 cm, 1.8 cm, 1.9 cm, or 2.0 cm.

5. The medical device according to claim 3, wherein the tether comprises a non-elastic material.

6. The medical device according to claim 5, wherein the tether comprises a width that is at least about 0.01 mm, 0.02 mm, 0.03 mm, 0.04 mm, 0.05 mm, 0.06 mm, 0.07 mm, 0.08 mm, 0.09 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1.0 mm, 1.5 mm, 2.0 mm, or 2.5 mm.

7. The medical device according to claim 2, wherein the tether tapers from the terminal end to the leading end.

8. The medical device according to claim 2, further comprising an attractive surface area on the puck.

9. The medical device according to claim 8, wherein the attractive surface area has a minimum diameter that is at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, or 30 mm.

10. The medical device according to claim 2, wherein the puck has a disk-shape.

11. The medical device according to claim 10, wherein the puck has a diameter of between about 1 mm and about 40 mm, between about 10 mm and about 30 mm, or between about 15 mm and about 25 mm.

12. The medical device according to claim 2, wherein the puck has a circumferential shape that varies in diameter.

13. The medical device according to claim 12, wherein the circumference shape has a maximum length of between about 1 mm and about 40 mm, between about 10 mm and about 30 mm, or between about 15 mm and about 25 mm.

14. The medical device according to claim 5, further comprising a guide that is magnetically attracted to the puck.

15. The medical device according to claim 14, wherein at least one of the guide and the puck comprises a magnet.

16. The medical device according to claim 14, wherein the puck and the guide comprise a magnet.

17. The medical device according to claim 16, wherein the guide comprises at least one of an electromagnet, a rare earth magnet, or a permanent magnet.

18. The medical device according to claim 16, wherein the surgical drain is a subdural drain.

19. A kit comprising:
a medical device comprising,
a tether;
a puck fixedly attached to a leading end of the tether;
a surgical drain operably attached to a terminal end of the tether; and
a guide that is magnetically attracted to the puck.

20. The kit according to claim 19, wherein the surgical drain comprises a distal end that is at least one of rounded and tapered.

21. The kit according to claim 20, wherein the tapered proximal end of the surgical drain has a length that is at least about 0.55 cm, 0.75 cm, 0.85 cm, 0.95 cm, 1.0 cm, 1.1 cm, 1.2 cm, 1.3 cm, 1.4 cm, 1.5 cm, 1.6 cm, 1.8 cm, 1.9 cm, or 2.0 cm.

22. The kit according to claim 20, wherein the tether comprises a non-elastic material.

23. The kit according to claim 22, wherein at least one of the guide and the puck comprises a magnet.

24. The kit according to claim 23, wherein the surgical drain is a subdural drain.

25. The kit according to claim 23, further comprising an introducer.

26. A method for the in vivo positioning a surgical drain within a cranium, the method comprising:
obtaining a kit comprising,
a tether with a puck fixedly attached to a leading end of the tether and the surgical drain having a distal end fixedly attached to a terminal end of the tether;
a guide magnetically attracted to the puck;
inserting the puck into a first hole in the cranium;
positioning the guide near the first hole to magnetically attract the puck through the tissue of the patient;
moving the guide over the cranium to direct the puck in vivo towards a second hole in the cranium, such that the tether attached to the puck is pulled through the first hole; and
moving the guide over the second hole to pull the puck and at least the leading end of the tether from the second hole.

27. The method according to claim 26, further comprising:
pulling the tether through the second hole until the surgical drain fixedly attached thereto is in proximity to the first hole;
inserting the distal end of the surgical drain into the first hole;
continuing to pull the leading end of the tether through the second hole to pull the surgical drain through the first hole;
pulling the surgical drain in vivo by pulling the tether through the second hole; and
pulling the distal end of the surgical drain through the second hole.

28. The method according to claim 27, further comprising:
removing the tether from the distal end of surgical drain; and
securing the proximal end of the surgical drain to maintain the in vivo position.

29. The method according to claim 28, wherein the surgical drain is a subdural drain.

30. The method according to claim 26, wherein the kit further comprises an introducer and the method further comprises inserting the puck through the first hole using the introducer to support the puck.

31. A method for the insertion and in vivo positioning of a subdural drain within a cranium, the method comprising:
obtaining a kit comprising,
a tether comprising a terminal end and a puck fixedly attached to a leading end of the tether;
a subdural drain comprising a distal end operably attachable to the tether;
a guide that is magnetically attracted to the puck;
inserting the puck into a first hole in the cranium;
positioning the guide near the first hole to magnetically attract the puck through the tissue of the patient;
moving the guide over the cranium to direct the puck in vivo towards a second hole in the cranium, such that the tether attached to the puck is pulled through the first hole; and
moving the guide over the second hole to pull the puck and at least the leading end of the tether from the second hole.

32. The method according to claim 31, further comprising:
operably attaching the distal end of the subdural drain to the terminal end of the tether extending from the first hole;
pulling the tether until the subdural drain operably attached thereto is in proximity to the first hole;
inserting the distal end of the subdural drain into the first hole;
continuing to pull the leading end of the tether through the second hole to pull the subdural drain through the first hole;
pulling the subdural drain in vivo by pulling the tether through the second hole; and
pulling the distal end of the subdural drain through the second hole.

33. The method according to claim 32, further comprising:
removing the tether from the distal end of subdural drain; and
securing the proximal end of the subdural drain to maintain the in vivo position.

34. The method according to claim 31, wherein the kit further comprises an introducer and the method further comprises inserting the puck into the first hole utilizing the introducer to support the puck.

35. The method according to claim 31, further comprising:
removing the puck from the leading end of the tether;
attaching the leading end of the tether to the distal end of the subdural drain;
pulling the terminal end of the tether through the first hole until the subdural drain is in proximity to the second hole;
inserting the subdural drain into the second hole;
continuing to pull the terminal end of the tether through the first hole to pull the surgical drain operably attached thereto through the second hole,
pulling the subdural drain in vivo by pulling the tether through the first hole; and
pulling the distal end of the subdural drain through the first hole.

36. The method according to claim 35, further comprising:
removing the tether from the distal end of the subdural drain; and
securing the proximal end of the subdural drain to maintain the in vivo position.

37. The method according to claim 35, wherein the kit further comprises an introducer and the method further comprises inserting the puck into the first hole utilizing the introducer to support the puck.

38. A medical device, configured to be positioned in vivo, comprising:
a tether;
a puck fixedly attached to a leading end of the tether; and
a surgical drain comprising a distal end operably attachable to the tether.

39. The medical device according to claim 38, further comprising a guide that is magnetically attracted to the puck.

40. The medical device according to claim 39, wherein at least one of the guide and the puck comprises a magnet.
